(19) United States
(12) Patent Application Publication
(10) Pub. No.: US 2025/0345808 A1
(43) Pub. Date: Nov. 13, 2025

(54) CENTRIFUGE FOR SEPARATING A FLUID FROM A ROCK SAMPLE

(71) Applicant: SAUDI ARABIAN OIL COMPANY, Dhahran (SA)

(72) Inventors: Hussain A. Jeshi, Qatif (SA); Atul Lalit Godbole, Dhahran (SA); Sultan M. Enezi, Dhahran (SA)

(73) Assignee: SAUDI ARABIAN OIL COMPANY, Dhahran (SA)

(21) Appl. No.: 18/658,045

(22) Filed: May 8, 2024

(51) Int. Cl. B04B 5/04; B04B 9/12; B04B 13/00; G01N 15/08; G01N 33/24

(52) U.S. Cl. CPC B04B 5/0414; B04B 9/12; G01N 15/0806; G01N 33/24; B04B 2013/006

(57) ABSTRACT

A centrifuge and methods are disclosed. The centrifuge may include a body having a chamber, an axle, and a motive element. The chamber has a circular cross-section in a plane perpendicular to an axis of rotation and defines an inward-facing inner wall surface. The axle extends into the chamber from an exterior position and is rotatable about the axis of rotation. The motive element is configured to rotate the axle. The centrifuge may further include a support member that includes a fixed member and a rotatable member. The fixed member is fixed to the inward-facing inner wall surface. The rotatable member is fixed to the axle within the chamber and rotatable relative to the fixed member about the axis of rotation. The centrifuge may still further include a sample housing disposed on the rotatable member. The sample housing includes a sample portion and a fluid portion.

### BACKGROUND

A centrifuge is configured to rotate one or more samples about an axis of rotation at an angular velocity to apply a centrifugal force to each of the one or more samples. Accordingly, constituents of each of the one or more samples separate based on density. The denser the constituent, the further away the constituent will separate from the residual sample relative to the axis of rotation. A centrifuge may be used in a variety of fields such as the medical industry, food industry, oil and gas industry, and even at home (e.g., laundry washing and drying machines). For a centrifuge to operate adequately, the centrifuge must be “balanced.” That is, the rotating portion of the centrifuge must remain level during rotation.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In general, in one aspect, embodiments relate to a centrifuge. The centrifuge includes a body having a chamber, an axle, and a motive element. The chamber has a circular cross-section in a plane perpendicular to an axis of rotation and defines an inward-facing inner wall surface. The axle extends into the chamber from an exterior position and is rotatable about the axis of rotation. The motive element is configured to rotate the axle. The centrifuge further includes a support member that includes a fixed member and a rotatable member. The fixed member is fixed to the inward-facing inner wall surface. The rotatable member is fixed to the axle within the chamber and rotatable relative to the fixed member about the axis of rotation. The centrifuge still further includes a sample housing disposed on the rotatable member. The sample housing includes a sample portion and a fluid portion.

In general, in one aspect, embodiments relate to a method. The method includes disposing a rock sample within the sample portion of the sample housing and disposing the sample housing on the rotatable member. The rock sample includes a fluid. The method further includes rotating, using the motive element, the rotatable member and the sample housing about the axis of rotation via the axle to apply a first centrifugal force to the rock sample. The method still further includes determining a first amount of the fluid within the fluid portion of the sample housing.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIG. 1A illustrates a side view of a centrifuge in accordance with one or more embodiments.

FIG. 1B illustrates a down-up view of a centrifuge in accordance with one or more embodiments.

FIG. 2A illustrates a side view of a centrifuge in accordance with one or more embodiments.

FIG. 2B illustrates a down-up view of a centrifuge in accordance with one or more embodiments.

FIG. 3 illustrates a side view of a centrifuge in accordance with one or more embodiments.

FIG. 4 illustrates a side view of a centrifuge in accordance with one or more embodiments.

FIG. 5 shows a free-body diagram of a sample housing in accordance with one or more embodiments.

FIG. 6 illustrates a motive element in accordance with one or more embodiments.

FIG. 7 illustrates a sample housing in accordance with one or more embodiments.

FIG. 8 describes a method in accordance with one or more embodiments.

FIG. 9 illustrates a computer system in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before,” “after,” “single,” and other such terminology. Rather, the use of ordinal numbers is to distinguish between the elements. By way of an example, a first element is distinct from a second element, and the first element may encompass more than one element and succeed (or precede) the second element in an ordering of elements.

It is to be understood that the singular forms “a,” “an,” and “the” include plural referents unless the context clearly dictates otherwise. Thus, for example, reference to “a support member” includes reference to one or more of such members.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

It is to be understood that one or more of the steps shown in the flowchart may be omitted, repeated, and/or performed in a different order than the order shown. Accordingly, the scope disclosed herein should not be considered limited to the specific arrangement of steps shown in the flowchart.

Although multiple dependent claims are not introduced, it would be apparent to one of ordinary skill that the subject matter of the dependent claims of one or more embodiments may be combined with other dependent claims.

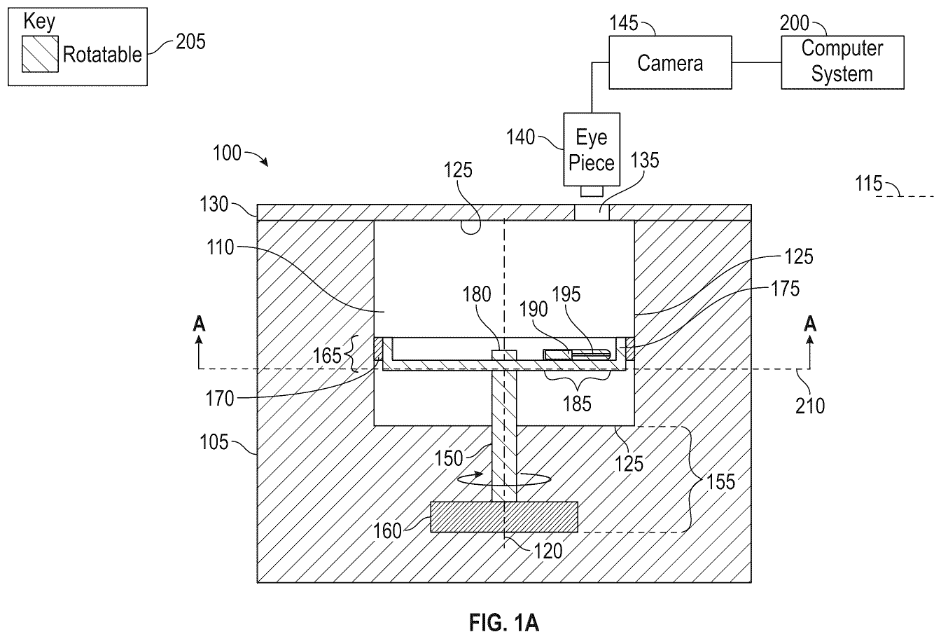

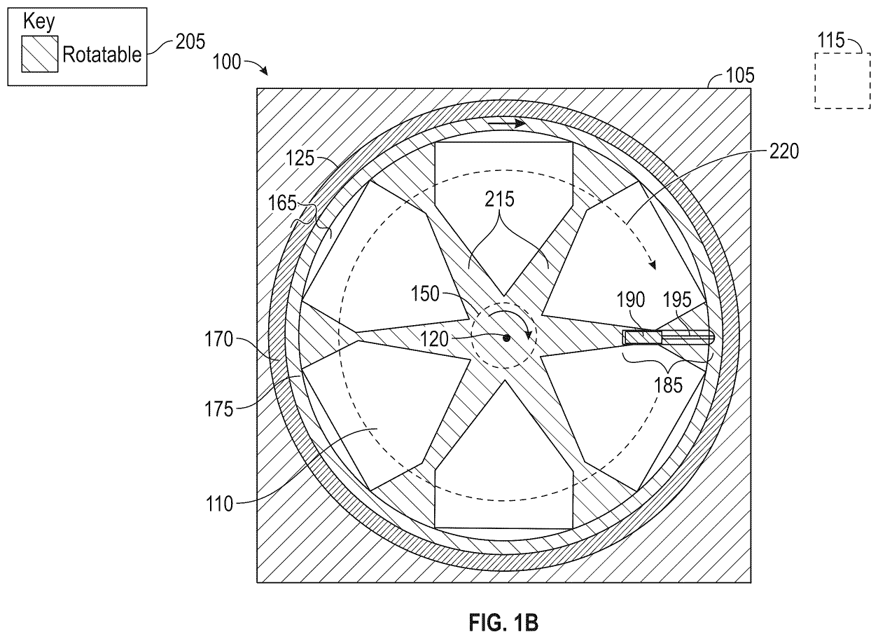

In the following description of FIGS. 1-9, any component described regarding a figure, in various embodiments disclosed herein, may be equivalent to one or more like-named components described regarding any other figure. For brevity, descriptions of these components will not be repeated regarding each figure. Thus, each and every embodiment of the components of each figure is incorporated by reference and assumed to be optionally present within every other figure having one or more like-named components.

Additionally, in accordance with various embodiments disclosed herein, any description of the components of a figure is to be interpreted as an optional embodiment which may be implemented in addition to, in conjunction with, or in place of the embodiments described regarding a corresponding like-named component in any other figure.

Embodiments of a centrifuge and methods of using the centrifuge are disclosed. The centrifuge may be configured to stabilize or support a sample housing while in operation (i.e., rotating).

In general, centrifuges are configured to rotate one or more samples about an axis of rotation at an angular velocity. Rotation of the one or more samples applies a centripetal force and, accordingly, a centrifugal force to each of the one or more samples to separate constituents of each sample by density. In some embodiments, each sample may include two or more fluid constituents. In other embodiments, each sample may include one or more fluid constituents (hereinafter simply “fluids”) and one or more solid constituents (hereinafter simply “solids”). The one or more fluids may be immiscible. Accordingly, two fluids within each sample may separate or a fluid may separate from a solid when the centrifugal force is applied to each sample. Further, two or more fluids separated from a solid may separate from one another.

To apply a constant centrifugal force to each of the one or more samples, the centrifuge needs to be “balanced” prior to rotation. That is, the one or more samples to be rotated within the centrifuge need to be evenly distributed by weight to ensure the one or more samples remain level while rotating. The longer the distance a sample is away from the axis of rotation, the more precise the balancing needs to be. In practice, a centrifuge may be balanced by placing two samples radially opposite to one another or by placing a weight radially opposite to each sample. If the centrifuge is not balanced prior to rotation, the centrifuge will be imbalanced and tilt or “wobble” during rotation. A wobble may result in inaccurate amounts of one or more fluids being separated or determined from a sample (especially at low angular velocities). Further, a wobble may place uneven loads on parts of the centrifuge thereby causing a requirement for frequent maintenance of the centrifuge.

The disclosed centrifuge is an improvement over other centrifuges as the disclosed centrifuge reduces the need to balance the centrifuge by implementing a support member. Accordingly, the disclosed centrifuge may be configured to rotate one or more samples placed anywhere within the disclosed centrifuge where the one or more samples need not be balanced. Additional benefits of the support member are described below following the description of the disclosed centrifuge.

Regarding the disclosed centrifuge as described in detail below, a person of ordinary skill in the art will appreciate that any element (i.e., feature) of the centrifuge described and/or illustrated may be combined with and/or modified by any other element of the centrifuge described and/or illustrated to configure the disclosed centrifuge. Note the elements illustrated in the figures may not be to scale. In some embodiments, the disclosed centrifuge may be a modified version of a commercial or stock centrifuge that is or was on the market. The commercial centrifuge may be, without limitation, a benchtop centrifuge, microcentrifuge, floor-standing centrifuge, large capacity centrifuge, STAT centrifuge, refrigerated centrifuge, high-speed centrifuge, and ultracentrifuge. In some embodiments, the commercial centrifuge may be used for medical purposes. However, the commercial centrifuge may be used in other fields.

FIG. 1A illustrates a side view of a centrifuge 100 in accordance with one or more embodiments. The centrifuge 100 includes a body 105. The body 105 may be any dimension and shape. The body 105 has a chamber 110. The chamber 110 may be any dimension and shape such that the chamber 110 has a circular cross-section in a plane 115 perpendicular to an axis of rotation 120. Accordingly, in some embodiments, the chamber 110 may be a cylinder or conical frustum (i.e., a frustum of a cone). FIG. 1A specifically illustrates the chamber 110 as a cylinder. The chamber 110 defines an inward-facing inner wall surface 125.

In some embodiments, the chamber 110 may be temporarily closed and/or sealed during operation of the centrifuge 100 by fixing a lid 130 over an opening of the chamber 110. Accordingly, the inward-facing inner wall surface 125 further extends along the lid 130. In some embodiments, the lid 130 may have a camera window 135 configured for an eye piece 140 of a camera 145 to view into a portion of the chamber 110.

The centrifuge 100 further includes an axle 150. The axle 150 extends into the chamber 110 from an exterior position 155. The axle 150 is rotatable about the axis of rotation 120. In practice, the axle 150 may be referred to as a shaft.

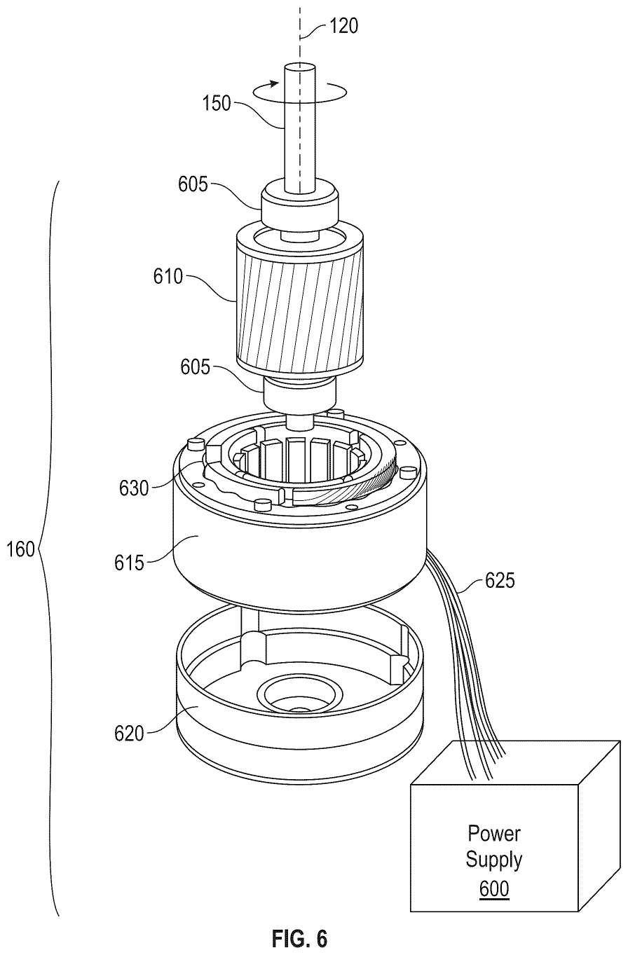

The centrifuge 100 further includes a motive element 160. The motive element 160 is configured to rotate the axle 150 about the axis of rotation 120. The motive element 160 is further discussed relative to FIG. 6.

The centrifuge 100 further includes the support member 165. The support member 165 includes a fixed member 170 and rotatable member 175. The fixed member 170 is fixed to the inward-facing inner wall surface 125 defined by the chamber 110. The fixed member 170 may be fixed to the inward-facing inner wall surface 125 using any means known to a person of ordinary skill in the art. The rotatable member 175 is fixed to the axle 150 within the chamber 110. The rotatable member 175 is rotatable relative to the fixed member 170 about the axis of rotation 120. Accordingly, the rotatable member 175 may nest or partially nest within the fixed member 170 as illustrated in FIG. 1A. In some embodiments, the rotatable member 175 may be fixed to an end 180 of the axle 150. In other embodiments, the end 180 of the axle 150 may extend beyond the portion of the rotatable member 175 fixed to the axle 150 and further into the chamber 110 as illustrated in FIG. 1A.

The centrifuge 100 further includes a sample housing 185. The sample housing 185 or portion thereof is disposed on and/or within the rotatable member 175 of the support member 165. The sample housing 185 is disposed on and/or within the rotatable member 175 using any means known to a person of ordinary skill in the art.

In practice, the sample housing 185 or portion thereof may be referred to as a vial or container. The sample housing 185 includes a sample portion 190 and fluid portion 195. In some embodiments, the sample housing 185 is disposed on and/or within the rotatable member 175 such that the sample portion 190 is closer to the axis of rotation 120 than the fluid portion 195 as illustrated in FIG. 1A. In some embodiments, the sample housing 185 is perpendicular to the axis of rotation 120 during rotation as illustrated in FIG. 1A. In other embodiments, the sample housing 185 swings from an angled position to a substantially-perpendicular position relative to the axis of rotation 120 during rotation.

In some embodiments, the centrifuge 100 may further still include a camera 145. The camera 145 may be configured to view or observe at least the fluid portion 195 of the sample housing 185 through the camera window 135 using the eye piece 140. In some embodiments, the camera 145 may be, without limitation, a digital camera and stroboscope. In some embodiments, the camera 145 may be configured to clearly view at least the fluid portion 195 while the sample housing 185 is stationary and/or rotating.

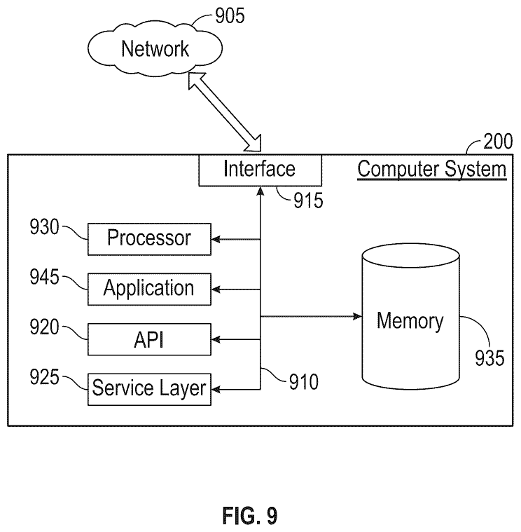

In some embodiments, the centrifuge 100 may further still include a computer system 200. The computer system 200 may be communicably coupled to the camera 145. The computer system 200 is discussed in detail relative to FIG. 9. In some embodiments, the computer system 200 may render an image of at least the fluid portion 195 captured by the camera 145. Accordingly, the computer system 200 may further render an image of any fluid collected within the fluid portion 195.

During operation of the centrifuge 100, the axle 150, rotatable member 175, and sample housing 185 rotate together about the axis of rotation 120 as illustrated by the shading and key 205.

FIG. 1B illustrates a down-up view of an A-A section 210 of the centrifuge 100 in accordance with one or more embodiments. The A-A section 210 is shown in FIG. 1A. As described relative to FIG. 1A and illustrated in FIG. 1B, the centrifuge 100 includes a body 105 having a chamber 110, support member 165, and sample housing 185. In these embodiments, the rotatable member 175 includes radially-extending arms 215. Though, FIG. 1B illustrates six radially-extending arms 215, any number of radially-extending arms 215 may be used. In some embodiments, a sample housing 185 may be disposed on each of the radially-extending arms 215.

During operation of the centrifuge 100, the axle 150, rotatable member 175, and sample housing 185 rotate together about the axis of rotation 120 to follow a rotational path 220.

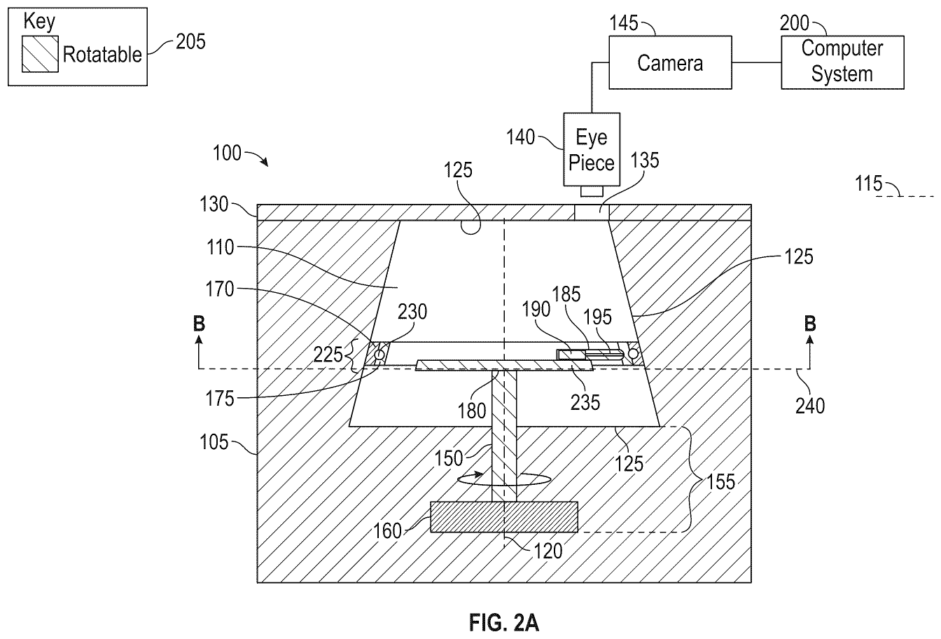

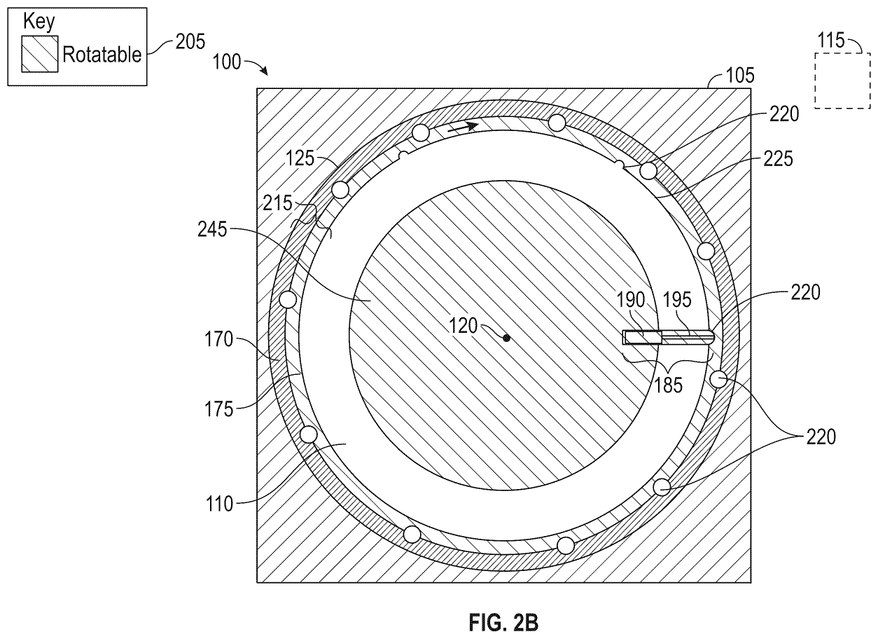

FIG. 2A illustrates a side view of the centrifuge 100 in accordance with one or more embodiments. In these embodiments, the chamber 110 is a conical frustum. In these embodiments, the support member 165 may be or include a bearing 225, specifically, a ball bearing. Accordingly, balls 230 are between the fixed member 170 and rotatable member 175 (or portions thereof) and configured to allow the rotatable member 175 to rotate relative to the fixed member 170 about the axis of rotation 120. Accordingly, in these embodiments, the fixed member 170 may be or include an outer race and the rotatable member 175 may be or include an inner race as illustrated in FIG. 2A. In some embodiments, the rotatable member 175 may include a table 235 parallel to the plane 115 as illustrated in FIG. 2A. In practice, the table may be referred to as a rotor or drum. In some embodiments, the table may be a stock rotor or rotor modified to accommodate the one or more samples or one or more sample housings. In some embodiments, the sample housing 185 may be disposed on and/or within the table 235 and/or inner race such that the sample housing 185 couples the table 235 and the inner race together. Accordingly, the rotatable member 175 (i.e., the table 235 and inner race) and sample housing 185 rotate together about the axis of rotation 120. Accordingly, the table 235 and inner race may or may not be fixed to one another.

FIG. 2B illustrates a down-up view of a B-B section 240 of the centrifuge 100 in accordance with one or more embodiments. The B-B section 240 is shown in FIG. 2A. In these embodiments, the table 235 of the rotatable member 175 is or includes a disc 245.

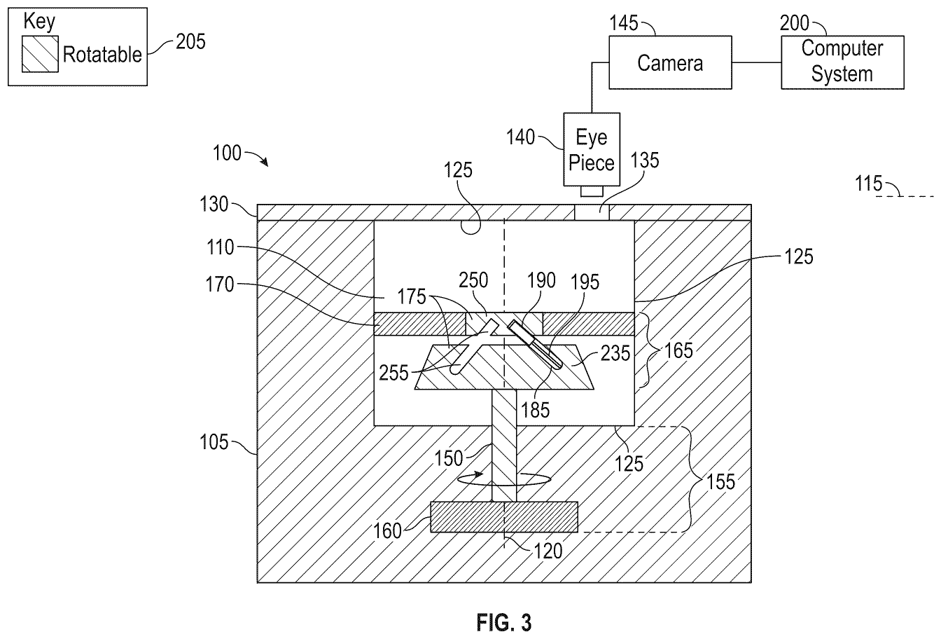

FIG. 3 illustrates a side view of the centrifuge 100 in accordance with one or more embodiments. In these embodiments, the table 235 of the rotatable member 175 is a conical frustum. In these embodiments, the remaining portion of the rotatable member 175 is a disc 250. In these embodiments, the rotatable member 175 (i.e., the table 235 and disc 250) includes one or more sample housing openings 255. In other embodiments, only the table 235 of the rotatable member 175 includes one or more sample housing openings 255. In still other embodiments, only the disc 250 includes one or more sample housing openings 255. In some embodiments, the disc 250 may be fixed to the table 235, axle 150, or both though none of these configurations are illustrated in FIG. 3. In other embodiments, the sample housing 185 may be disposed on and/or within the table 235 and disc 250 such that the sample housing 185 couples the table 235 and disc 250 together as illustrated in FIG. 3.

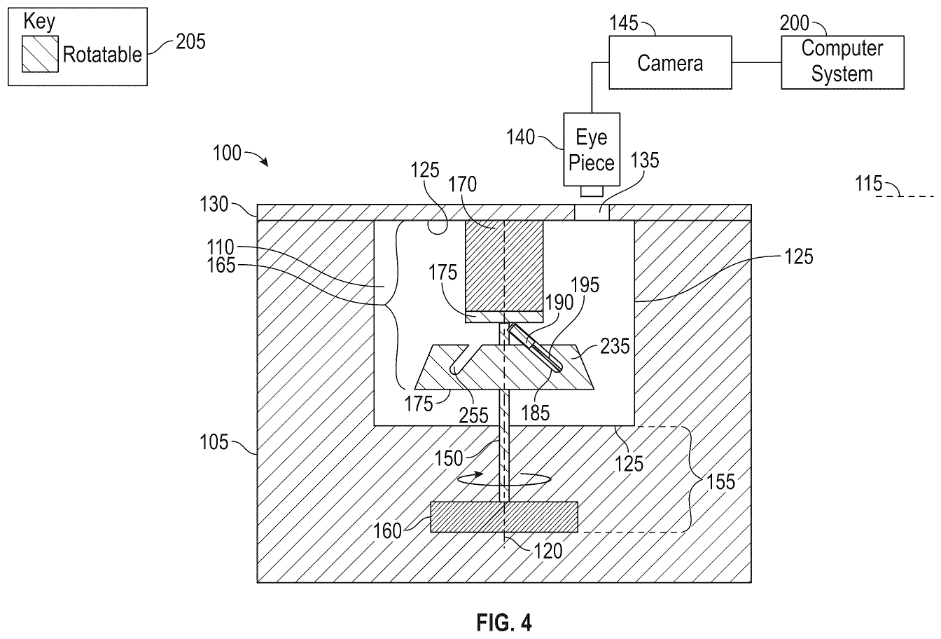

FIG. 4 illustrates a side view of the centrifuge 100 in accordance with one or more embodiments. In these embodiments, the fixed member 170 and rotatable member 175 of the support member 165 are stacked one on top of the other. In these embodiments, the fixed member 170 is fixed to the inward-facing inner wall surface 125 immediately next to the lid 130 as illustrated in FIG. 4. In some embodiments, though not shown in FIG. 4, the fixed member 170 may be alternatively or additionally fixed to the inward-facing inner wall surface 125 immediately next to the body 105. In some embodiments, each of the fixed member 170 and rotatable member 175 may be, or include, a race or disc though other shapes could be used.

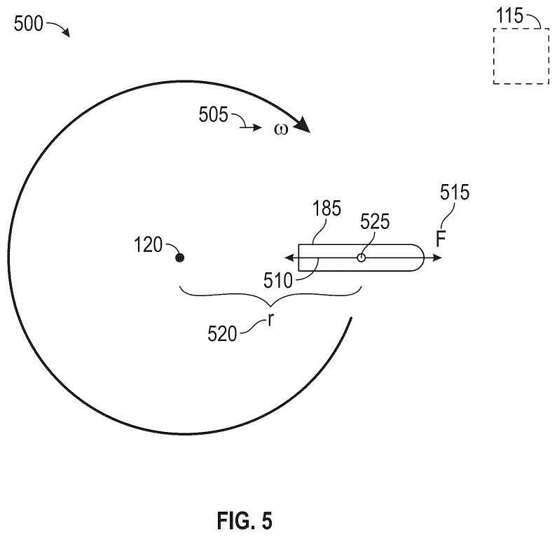

FIG. 5 illustrates a free-body diagram 500 of the sample housing 185 in accordance with one or more embodiments. During operation of the centrifuge 100, the rotatable member 175 and sample housing 185 rotate about the axis of rotation 120 at an angular velocity ω 505. Accordingly, a centripetal force 510 is applied to the sample housing 185 (and the sample within the sample housing 185) towards the axis of rotation 120. Further, a centrifugal force F 515 is observed opposite the centripetal force 510 relative to the sample housing 185 (and the sample within the sample housing 185). The relationship between the angular velocity ω 505 and centrifugal force F 515 may be given as:

$$F = m {ω}^{2} r , \qquad Equation ( l )$$

where m is the mass of the sample housing 185 (including the mass of the sample within the sample housing 185) and r is the distance 520 from the axis of rotation 120 to the center of mass 525 of the sample housing 185 (including the mass of the sample within the sample housing 185). Note the centrifugal force F 515 is an inertial force sometimes referred to as a fictitious or pseudo force. According to Equation (1), the longer the distance r 520 and/or the greater the angular velocity ω 505, the greater the centrifugal force F 515.

FIG. 6 illustrates a motive element 160 and axle 150 in accordance with one or more embodiments. In some embodiments, the motive element 160 may be housed in the body 105 of the centrifuge 100 as FIGS. 1A, 2A, 3, and 4 illustrate. In other embodiments, the motive element 160 or portion thereof may be outside the body 105 of the centrifuge 100. In some embodiments, the motive element 160 may be a motor, such as, without limitation, an alternating current (AC) motor, direct current (DC) motor, inverter motor, stepping motor, and switched reluctance motor. In these embodiments, the motive element 160 may be connected to a power supply 600. In other embodiments, the motive element 160 may be any device configured to rotate the axle 150 about the axis of rotation 120, such as a manual device.

FIG. 6 illustrates the motive element 160 as a generic motor. The motive element 160 may include bearings 605, a rotor 610, a stator 615, a bracket 620, and lead wire 625 connected to the power supply 600. Any type of bearing 605, stator 614, bracket 620, lead wire 625, and power supply 600 may be used to configure the motive element 160. In brief, the stator 615 generates force capable of rotating the rotor 610, supported by the bearings 605, and axle 150 about the axis of rotation 120. In some embodiments, the stator 615 may generate force using winding 630 (i.e., magnetic wire). To do so, electricity is supplied to the winding 630 from the power supply 600 via the lead wire 625 to generate a magnetic field. In other embodiments, the stator 615 may be an inductor stator or permanent magnet stator. Accordingly, the motive element 160 may be configured to rotate the rotor 610, axle 150, rotatable member 175, and sample housing 185 (including a sample) about the axis of rotation 120 at prescribed angular velocities 505 to apply prescribed centrifugal forces 515 to the sample housing 185 (including the sample).

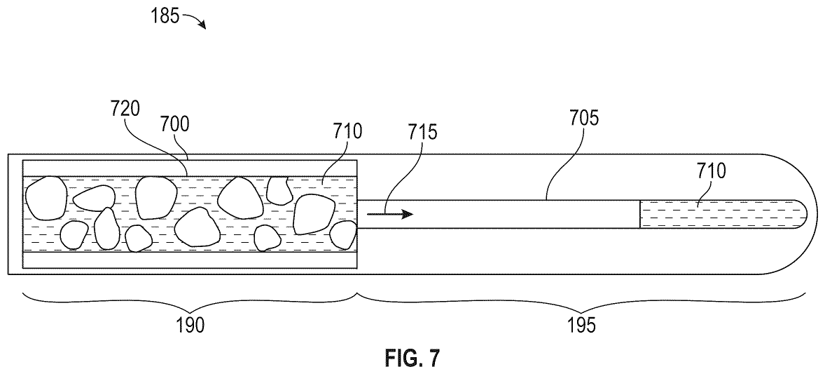

FIG. 7 illustrates the sample housing 185 in accordance with one or more embodiments. The sample housing 185 includes a sample portion 190 and fluid portion 195. In some embodiments, the sample portion 190 may include a sample chamber 700. In some embodiments, the fluid portion 195 may include a fluid chamber 705. The fluid chamber 705 may be a transparent tube made of sapphire. In these embodiments, the sample chamber 700 and fluid chamber 705 may be in fluid communication with one another (i.e., be fluidly coupled). Further, in these embodiments, the sample chamber 700 and fluid chamber 705 may be in one-way fluid communication with one another. For example, once fluid 710 initially in the sample chamber 700 is displaced and flows into the fluid chamber 705 as illustrated by the direction of fluid travel 715, the fluid 710 in the fluid chamber 705 may be unable to return to the sample chamber 700.

In some embodiments, a rock sample 720 may be disposed within the sample portion 190 or sample chamber 700 of the sample housing 185 as illustrated in FIG. 7. The rock sample 720 may include or be saturated with one or more fluids 710. Further, the rock sample 720 may take any dimension and shape. Following disposition of the rock sample 720 within the sample portion 190 or sample chamber 700, the sample housing 185 may be disposed on and/or within the rotatable member 175 of the centrifuge 100 and rotated. During rotation, the fluid 710 within the rock sample 720 may flow into the fluid portion 195 or fluid chamber 705. Though the sample housing 185 illustrated in FIG. 7 is described relative to housing a rock sample 720, a person of ordinary skill in the art will appreciate that other types of samples, solids and/or fluids, may be disposed in the sample portion 190 or sample chamber 700 of the sample housing 185 and rotated in the centrifuge 100 without departing from the scope of the disclosure.

In summary, implementation of the support member 165 within the centrifuge 100 offers numerous benefits, which include the following. The rotatable member 175, and thus each rock sample 720 disposed in each sample housing 185, are inherently balanced (or nearly balanced) and will not wobble during rotation by virtue of the support member 165. Accordingly, there is little-to-no need to balance the rotatable member 175 using one or more rock samples 720 or weights. Each rock sample 720 disposed in each sample housing 185 will be stable. Accordingly, the sample housing 185 is more likely to remain sealed and, thus, is less likely to unintentionally tilt and/or open such that the fluid 710 within or separated from the rock sample 720 or portion thereof spills during rotation. The amount of separated constituent(s) (e.g., fluid 710) from each rock sample 720 may be more precisely determined. For example, a contact line or floating disc within the fluid portion 195 of the sample housing 185 that separates immiscible fluids may remain horizontal or vertical (i.e., not tilted) such that the camera 145 clearly images each of the separated immiscible fluids. Noise and/or vibration caused by the rotating parts of the centrifuge 100 may be reduced. For example, a radially-extending arm 215 may not bend as it might on other centrifuges due to, for example, the weight of the rock sample 720 and/or sample housing 185. Accordingly, the chance of instrument damage, such as failing parts, may be reduced. Accordingly, the centrifuge 100 may be safer to use.

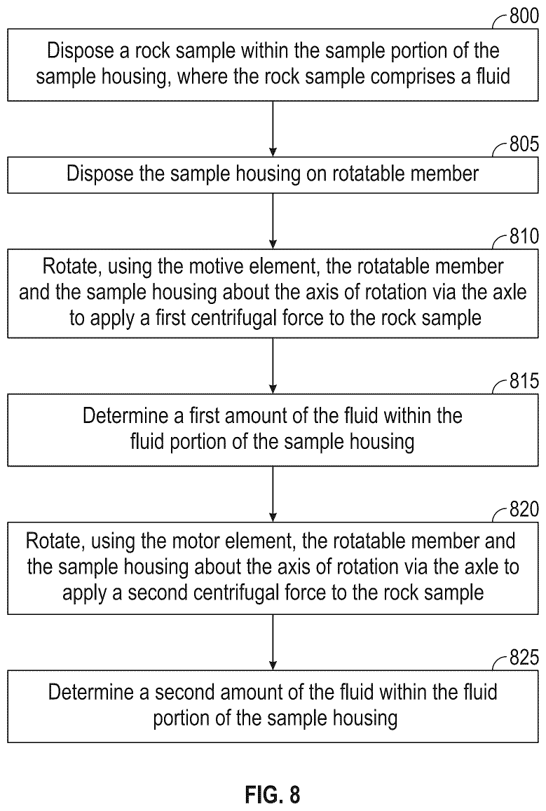

FIG. 8 describes a method in accordance with one or more embodiments. In step 800, a rock sample 720 is disposed with a sample portion 190 of the sample housing 185 as illustrated in FIG. 7. The rock sample 720 includes or is saturated with a fluid 710.

In step 805, the sample housing 185 that includes the rock sample 720 is disposed on and/or within the rotatable member 175 of the centrifuge 100 as illustrated in FIGS. 1A, 1B, 2A, 2B, 3, and 4.

In step 810, the rotatable member 175 and sample housing 185 that includes the rock sample 720 is rotated using the motive element 160 via the axle 150 at a first angular velocity. Accordingly, a first centrifugal force is applied to the rock sample 720 during rotation. During rotation, the fluid 710 or portion thereof within the rock sample 720 may be displaced or separated from the solids of the rock sample 720 and travel to and be collected by the fluid portion 195 of the sample housing 185.

In step 815, a first amount of fluid is determined within the fluid portion 195 of the sample housing 185. In some embodiments, the first amount of fluid may be a first amount of fluid once steady state or equilibrium is reached. In some embodiments, the first amount of fluid may be determined using a camera 145, such as a stroboscope, configured to view at least the fluid portion 195 of the sample housing 185. In some embodiments, the camera 145 may view at least the fluid portion 195 during rotation or once rotation has stopped. In some embodiments, a computer system 200 communicably coupled to the camera 145 may render an image of at least the fluid portion 195 and determine the first amount of fluid within the fluid portion 195.

In some embodiments, steps 820 and 825 may be performed.

In step 820, the rotatable member 175 and sample housing 185 that includes the residual rock sample 720 and separated fluid 710 is rotated using the motive element 160 via the axle 150 at a second angular velocity. In some embodiments, the second angular velocity may be applied immediately following the first angular velocity without pause or break in rotation. Accordingly, a second centrifugal force is applied to the rock sample 720. During rotation, residual fluid 710 or portion thereof within the residual rock sample 720 may be displaced from the solids of the rock sample 720 and travel to and be collected by the fluid portion 195 of the sample housing 185 that collected the first amount of fluid. In some embodiments, the second centrifugal force is greater than the first centrifugal force.

In step 825, a second amount of fluid is determined within the fluid portion 195 of the sample housing 185. In some embodiments, the second amount of fluid may be a second amount of fluid once steady state or equilibrium is reached. In some embodiments, the second amount of fluid may be determined using a camera 145, such as a stroboscope, configured to view at least the fluid portion 195 of the sample housing 185. In some embodiments, the camera 145 may view at least the fluid portion 195 during rotation or once rotation has stopped. In some embodiments, a computer system 200 communicably coupled to the camera 145 may render an image of at least the fluid portion 195 and determine the second amount of fluid within the fluid portion 195.

In some embodiments, wettability, capillary pressure, and/or relative permeability of the rock sample 720 may be determined following steps 800, 805, 810, 815, 820, and 825 and, in some embodiments, steps 820 and 825 repeated at greater and greater centrifugal forces 515. To do so, wettability, capillary pressure, and/or relative permeability may be determined based, at least in part, on the centrifugal forces 515 applied to the rock sample 720 and the amount of fluid within the fluid portion 195 after each centrifugal force 515 is applied. Wettability, capillary pressure, and/or relative permeability may be considered special core analysis data or SCAL data, among other data. SCAL data may be crucial for informing hydrocarbon reservoir simulations and hydrocarbon reservoir descriptions.

Wettability is the preference of a solid to contact a liquid or gas (collectively “fluid”). Wettability is also known as the wetting phase. In some embodiments, wettability of a rock sample 720, in the form of wettability number or index, may be determined using the United States Bureau of Mines (USBM) test as described in Donaldson, Erle C., Rex D. Thomas, and Philip B. Lorenz. “Wettability determination and its effect on recovery efficiency.” Society of Petroleum Engineers Journal 9.01 (1969): 13-20. To do so, in some embodiments, a rock sample 720 may be reduced to its initial water saturation s_{w} and aged in crude oil at hydrocarbon reservoir conditions. The rock sample 720 may then be placed in a brine-displacing-oil system and oil-displacing-brine system and rotated in the centrifuge 100 at a range of angular velocities 505 to apply a range of centrifugal forces 515 to the system. Because each angular velocity 505 corresponds to a capillary pressure, drainage and imbibition capillary pressure displacement curves may be determined and the area under each curve used to determine the wettability number w where:

$$w = log \frac{{A}_{dr}}{{A}_{imb}} , \qquad Equation ( 2 )$$

where A_{dr} is the area under the drainage capillary pressure displacement curve and A_{imb} is the area under the imbibition capillary pressure displacement curve for a rock sample 720. The wettability number w is positive for water-wet samples and negative for oil-wet samples. Please refer to Donaldson for a detailed discussion of determining wettability of the rock sample 720. Further, note that other methods, such as the Amott test, may be used to determine wettability of the rock sample 720 without departing from the scope of the disclosure.

In some embodiments, capillary pressure of the rock sample 720 may be determined using the method described by Hassler, G. L., and E. Brunner. “Measurement of capillary pressures in small core samples.” Transactions of the AIME 160.01 (1945): 114-123. In brief, to perform Hassler and Brunner's method, steps 820 and 825 may be repeated to apply continually increasing centrifugal forces 515 to the rock sample 720 in series. The term z, which represents capillary pressure, may be determined for the rock sample 720 at each angular velocity 505 as:

$$z = \frac{1}{2} ρ {ω}^{2} ( {r}_{2}^{2} - {r}_{1}^{2} ) , \qquad Equation ( 3 )$$

where ρ is the density of the fluid 710 within the rock sample 720, w is the angular velocity 505 of the rock sample 720, and r₁ and r₂ are the distances of the two ends of the rock sample 720 from the axis of rotation 120, respectively (see equation [6] of Hassler and Brunner). Please refer to Hassler and Brunner for a detailed discussion of determining capillary pressure of the rock sample 720. Further, note that other methods may be used to determine capillary pressure of the rock sample 720 without departing from the scope of the disclosure.

Relative permeability of the rock sample 720 may be determined using any method known to a person of ordinary skill in the art. In some embodiments, relative permeability of the rock sample 720 may be determined using the method described by O'Meara Jr, D. J., and J. G. Crump. “Measuring capillary pressure and relative permeability in a single centrifuge experiment.” SPE Annual Technical Conference and Exhibition. SPE, 1985. In other embodiments, relative permeability of the rock sample 720 may be determined using the method described by Hagoort, Jo. “Oil recovery by gravity drainage.” Society of Petroleum Engineers Journal 20.03 (1980): 139-150. However, a person of ordinary skill in the art will appreciate that other methods to determine relative permeability may be used.

FIG. 8 illustrates a computer system 200 in accordance with one or more embodiments. In some embodiments, the computer system 200 may be communicably coupled to the camera 145. Accordingly, the computer system 200 may render an image of at least the fluid portion 195 of the sample housing 185 and determine an amount of fluid within the fluid portion 195 after each of one or more centrifugal forces 515 is applied to the rock sample 720 disposed in the sample housing 185.

The computer system 200 is intended to depict any computing device such as a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer system 200 may include an input device, such as a keypad, keyboard, touch screen, or other device that can accept user information, and an output device that displays information, including digital data, visual or audio information (or a combination of both), or a graphical user interface (GUI).

The computer system 200 can serve in a role as a client, network component, server, database, or any other component (or a combination of roles) of a computer system 200 as required for use with the camera 145. The illustrated computer system 200 is communicably coupled with a network 905. In some implementations, one or more components of each computer system 200 may be configured to operate within environments, including cloud-computing-based, local, global, or other environment (or a combination of environments).

At a high level, the computer system 200 is an electronic computing device operable to receive, transmit, process, store, and/or manage data and information associated with the disclosed centrifuge 100 and methods. According to some implementations, the computer system 200 may also include or be communicably coupled with an application server, e-mail server, web server, caching server, streaming data server, business intelligence (BI) server, or other server (or a combination of servers).

The computer system 200 can receive requests over the network 905 from other computer systems 200 or another client application and respond to the received requests by processing the requests appropriately. In addition, requests may also be sent to the computer system 200 from internal users (for example, from a command console or by other appropriate access method), external or third-parties, other automated applications, as well as any other appropriate entities, individuals, systems, or computer systems 200.

Each of the components of the computer system 200 can communicate using a system bus 910. In some implementations, any or all of the components of each computer system 200, both hardware or software (or a combination of hardware and software), may interface with each other or the interface 915 (or a combination of both) over the system bus 910 using an application programming interface (API) 920 or a service layer 925 (or a combination of the API 920 and service layer 925. The API 920 may include specifications for routines, data structures, and object classes. The API 920 may be either computer-language independent or dependent and refer to a complete interface, a single function, or even a set of APIs. The service layer 925 provides software services to each computer system 200 or other components (whether or not illustrated) that are communicably coupled to each computer system 200. The functionality of each computer system 200 may be accessible for all service consumers using this service layer 925. Software services, such as those provided by the service layer 925, provide reusable, defined business functionalities through a defined interface. For example, the interface may be software written in JAVA, C++, or other suitable language providing data in extensible markup language (XML) format or another suitable format. While illustrated as an integrated component of each computer system 200, alternative implementations may illustrate the API 920 or the service layer 925 as stand-alone components in relation to other components of each computer system 200 or other components (whether or not illustrated) that are communicably coupled to each computer system 200. Moreover, any or all parts of the API 920 or the service layer 925 may be implemented as child or sub-modules of another software module, enterprise application, or hardware module without departing from the scope of this disclosure.

The computer system 200 includes an interface 915. Although illustrated as a single interface 915 in FIG. 9, two or more interfaces 915 may be used according to particular needs, desires, or particular implementations of each computer system 200. The interface 915 is used by each computer system 200 for communicating with other systems in a distributed environment that are connected to the network 905. Generally, the interface 915 includes logic encoded in software or hardware (or a combination of software and hardware) and operable to communicate with the network 905. More specifically, the interface 915 may include software supporting one or more communication protocols associated with communications such that the network 905 or interface's hardware is operable to communicate physical signals within and outside of the illustrated computer system 200.

The computer system 200 includes at least one processor 930. Generally, a processor 930 executes any instructions, algorithms, methods, functions, processes, flows, and procedures as described above. A processor 930 may be a central processing unit (CPU) and/or a graphics processing unit (GPU).

The computer system 200 also includes a memory 935 that stores data and software for the computer system 200 or other components (or a combination of both), such as the camera 145, that can be connected to the network 905. Although illustrated as a single memory 935 in FIG. 9, two or more memories may be used according to particular needs, desires, or particular implementations of the computer system 200 and the described functionality. While memory 935 is illustrated as an integral component of each computer system 200, in alternative implementations, memory 935 can be external to the computer system 200.

The application 945 is an algorithmic software engine providing functionality according to particular needs, desires, or particular implementations of the computer system 200, particularly with respect to functionality described in this disclosure. For example, application 945 can serve as one or more components, modules, applications, etc. Further, although illustrated as a single application 945, the application 945 may be implemented as multiple applications 945 on each computer system 200. In addition, although illustrated as integral to each computer system 200, in alternative implementations, the application 945 can be external to each computer system 200.

There may be any number of computer systems 200, such as computer clusters, associated with, or external to, the computer system 200, where each computer system 200 communicates over network 905. Further, the term “client,” “user,” and other appropriate terminology may be used interchangeably as appropriate without departing from the scope of this disclosure. Moreover, this disclosure contemplates that many users may use the computer system 200, or that one user may use multiple computer systems 200.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed is:

1. A centrifuge comprising:
a body having a chamber,
wherein the chamber has a circular cross-section in a plane perpendicular to an axis of rotation, and
wherein the chamber defines an inward-facing inner wall surface;
an axle extending into the chamber from an exterior position,
wherein the axle is rotatable about the axis of rotation;
a motive element configured to rotate the axle;
a support member comprising a fixed member and a rotatable member,
wherein the fixed member is fixed to the inward-facing inner wall surface, and
wherein the rotatable member is fixed to the axle within the chamber and rotatable relative to the fixed member about the axis of rotation; and
a sample housing disposed on the rotatable member,
wherein the sample housing comprises a sample portion and a fluid portion.

2. The centrifuge of claim 1, wherein the rotatable member comprises a disc.

3. The centrifuge of claim 1, wherein the rotatable member comprises a plurality of radially-extending arms.

4. The centrifuge of claim 3, wherein the sample housing is disposed on one of the plurality of radially-extending arms.

5. The centrifuge of claim 1, wherein the rotatable member comprises a table, and wherein the table is fixed to the axle within the chamber.

6. The centrifuge of claim 1, wherein the sample housing extends into the rotatable member.

7. The centrifuge of claim 1, wherein the sample housing is disposed such that the sample portion is closer to the axis of rotation than the fluid portion, and
wherein the sample housing is perpendicular to the axis of rotation.

8. The centrifuge of claim 1, wherein the sample portion comprises a sample chamber,
wherein the fluid portion comprises a fluid chamber, and
wherein the sample chamber and the fluid chamber are in fluid communication.

9. The centrifuge of claim 1, further comprising a camera configured to view the fluid portion.

10. The centrifuge of claim 9, further comprising a computer system communicably coupled to the camera.

11. The centrifuge of claim 1, wherein the support member comprises a bearing.

12. The centrifuge of claim 1, wherein the fixed member comprises an outer race.

13. The centrifuge of claim 1, wherein the rotatable member comprises an inner race.

14. A method of separating, using a centrifuge, a fluid from within a sample, the centrifuge comprising:
a body having a chamber,
wherein the chamber has a circular cross-section in a plane perpendicular to an axis of rotation, and
wherein the chamber defines an inward-facing inner wall surface;
an axle extending into the chamber from an exterior position,
wherein the axle is rotatable about the axis of rotation;
a motive element configured to rotate the axle;
a support member comprising a fixed member and a rotatable member,
wherein the fixed member is fixed to the inward-facing inner wall surface, and
wherein the rotatable member is fixed to the axle within the chamber and rotatable relative to the fixed member about the axis of rotation;
a sample housing disposed on the rotatable member,
wherein the sample housing comprises a sample portion and a fluid portion;
wherein the method comprises:
disposing a rock sample within the sample portion of the sample housing,
wherein the rock sample comprises a fluid;
disposing the sample housing on the rotatable member;
rotating, using the motive element, the rotatable member and the sample housing about the axis of rotation via the axle to apply a first centrifugal force to the rock sample; and
determining a first amount of the fluid within the fluid portion of the sample housing.

15. The method of claim 14, wherein determining the first amount of the fluid comprises using a camera coupled to a computer system, and
wherein the camera is configured to view the fluid portion.

16. The method of claim 14, further comprising:
rotating, using the motive element, the rotatable member and the sample housing about the axis of rotation via the axle to apply a second centrifugal force to the rock sample; and
determining a second amount of the fluid within the fluid portion of the sample housing.

17. The method of claim 16, wherein the second centrifugal force is greater than the first centrifugal force.

18. The method of claim 16, further comprising determining wettability of the rock sample based, at least in part, on the first amount of fluid, the first centrifugal force, the second amount of fluid, and the second centrifugal force.

19. The method of claim 16, further comprising determining capillary pressure of the rock sample based, at least in part, on the first amount of fluid, the first centrifugal force, the second amount of fluid, and the second centrifugal force.

20. The method of claim 16, further comprising determining relative permeability of the rock sample based, at least in part, on the first amount of fluid, the first centrifugal force, the second amount of fluid, and the second centrifugal force.
